(19) United States
(12) Patent Application Publication
(10) Pub. No.: US 2002/0177691 A1
(43) Pub. Date: Nov. 28, 2002

(54) Trans inteins for protein domain shuffling and biopolymerization

(76) Inventors: Charles P. Scott, Narberth, PA (US); Stephen J. Benkovic, State College, PA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/103,467

(22) Filed: Mar. 20, 2002

Related U.S. Application Data
(60) Provisional application No. 60/277,402, filed on Mar. 20, 2001.

(51) Int. Cl. C07K 14/00; G01N 33/53; C12P 21/04; C12N 5/06

(52) U.S. Cl. 530/350; 435/7.1; 435/69.7; 435/320.1; 435/325

(57) ABSTRACT

This invention provides improved methods of protein engineering, reagents useful therewith, and combinatorial libraries of chimeric proteins produced independent of amino acid or nucleotide sequence homology.

This application claims priority to U.S. provisional application Serial No. 60/277,402, filed Mar. 20, 2001.

This application was supported by a grant from the National Institutes of Health, No. GM 19891. The government may have certain rights in this invention.

### BACKGROUND OF THE INVENTION

1. Field of the Invention

This invention relates to genetic engineering and production of proteins using genetic engineering techniques. The invention particularly relates to production of polymeric proteins, particularly polymeric proteins comprising repeating units of specific amino acid sequence motifs. The invention specifically provides reagents and methods for producing such proteins in recombinant cells using a multiplicity of genetic constructs comprising at least one sequence domain or motif of a protein wherein the nucleic acid sequence encoding the sequence domain or motif is operably linked to an amino or carboxyl portion of a trans-intein. According to the invention, the recombinant protein is produced in the cell or in the cell culture medium by post-translational polymerization of sequence domains or motifs using specific recognition of one portion of a trans-intein with its cognate portion of the intein. Recombinant proteins, recombinant expression constructs, recombinant cells, and libraries of recombinant constructs encoding fragments, including random fragments, of cellular proteins operably linked to an amino or carboxyl portion of a trans-intein are also provided by the invention.

2. Background of the Related Art

Genetic engineering and recombinant DNA technology have enabled production of a wide range of naturally-occurring proteins. However, some proteins, particularly those having certain sequence domains or motifs, and most particularly those having repeated copies of sequence domains or repeats, are difficult to express using conventional recombinant DNA techniques. This is because certain of these domains or repeats are of necessity encoded by repetitive DNA sequences, thus providing opportunities for genetic recombination that alter the DNA sequence, for example, by increasing or decreasing the number of repeats or shifting the reading frame of the translated protein. This results in genetic instability and sub-optimal recombinant protein production.

In addition, protein mutagenesis methods have been recognized in the art as being useful for identifying sequences in proteins associated with particular activities or substrate specificities. Such mutagenesis techniques have proven to require extensive experimentation to implement and to be unpredictable for producing mutant proteins retaining the same or altered activities. It is now recognized that certain biochemical activities (such as ATP binding and ATPase activity) in a variety of different naturally-occurring proteins are mediated by particular amino acid sequence motifs (such as ATP binding cassette motifs). It has also been shown by some of the instant inventors that proteins having related function (but, for example, that are derived from different species) can be recombined to produce novel proteins having activities related to but different from either of the parent proteins (see, for example, Lutz et al., 2001, Nucleic Acids Res. 29: E16). Methods known in the art useful in producing such “directed chimeras” include incremental truncation for the creation of hybrid enzymes (“ITCHY;” Ostermeier et al., 1999, Bioorg. Med. Chem. 7: 2139-2144 and International Application, Publication No. WO 01/75158, published Oct. 11, 2001 and incorporated by reference) and a variant combining ITCHY with DNA shuffling protocols (termed “SCRATCHY;” Lutz et al., 2001, Proc. Natl. Acad. Sci. USA 98: 11248-11253). However, these techniques have been limited to making particular variants of particular proteins or novel chimeras between known and related proteins.

Classes of reagents useful in both the ITCHY and SCRATCHY protocols are cis- and trans-inteins. Interns are a class of genetic element encoding a protein having self-recognition and autocatalytic properties. A cis-intein is an internal peptide sequence of a protein precursor that is spliced out by transpeptidation during posttranslational processing to form a mature protein (Perler et al., 2000, Curr. Opin. Biotechnol. 11: 377-383). Cis-inteins function post-translationally to covalently link protein or peptide fragments that are joined to the amino terminus of the intein with protein or peptide fragments that are joined to the carboxyl terminus of the intein, leaving a cysteine residue at the junction. While useful for protein affinity purification (Chong et al., 1997, Gene 192(2): 271-81) and expressed protein ligation (Severinov et al., 1998, J. Biol. Chem. 273: 16205-16209) in the canonical configuration, and for producing cyclic proteins and peptides in a permuted configuration (Scott et al., 1999, Proc. Natl. Acad. Sci. USA 96: 13638-43), there are limitations to the use of cis-inteins that are recognized in the art (see, for example, Iwai et al., 2001, J. Biol. Chem. 276: 16548-16554). Trans-inteins similarly join post-translationally different protein or peptide fragments covalently linked to cognate portions of the trans-inteins; unlike cis-inteins, however, the cognate portion of trans-inteins are not covalently linked to one another and must associate or bind to one another in the recombinant cell or in solution to effect covalent linkage of the protein or peptide fragments linked to each portion of the trans-intein (see Ozawa et al., 2001, Anal. Chem. 73: 2516-2521). Trans-inteins thus have the capacity to produce chimeric proteins by the combination of different protein or peptide fragments to different cognate portions of the intein, rather than to either end of a single intein as is the case with cis-inteins.

Thus, there remains a need in the art for producing proteins, particularly polymeric proteins comprising repeated sequence domains or motifs, that cannot be advantageously or reliably produced using conventional recombinant protein production techniques. There is also a need in the art to develop more efficient and effective methods for producing chimeric proteins having improved or unique properties or activities compared with naturally-occurring proteins.

### SUMMARY OF THE INVENTION

The present invention provides reagents and methods for overcoming the limitations in the art associated with recombinant production of chimeric and polymeric proteins such as intracellular recombination and permits more efficient production of recombinant proteins. The present invention provides methods for producing chimeric proteins, producing combinatorial protein libraries, and engineering trans-inteins from cis-inteins. The present invention also provides recombinant expression constructs, host cells, cis- and trans-inteins, and recombinant methods for producing polynucleotides and polypeptides. The invention also provides methods and reagents for producing recombinant libraries, preferably random fragment libraries and most preferably embodiments of said libraries wherein each protein fragment encoding sequence is operably linked to a portion of an intein.

In one aspect, the present invention provides improved methods of protein engineering, most preferably non-homology dependent protein engineering, wherein combinatorial libraries of chimeric polypeptides are post-translationally recombined via the actions of trans-inteins. In the practice of the methods of the invention in this aspect, random protein fragment-encoding nucleic acids are produced, by randomly-primed cDNA synthesis from cellular RNA or by incremental truncation of protein-encoding domains (Ostermeier et al., 1999, Bioorg. Med. Chem. 7: 2139-2144) and cloned into recombinant expression constructs so that the sequences are operably linked to an amino- or carboxyl-terminal portion of a trans-intein. In preferred embodiments, the recombinant expression construct is a modified retroviral vector that can be used to produce virus infectious in any advantageous mammalian cell type. Other preferred embodiments include introducing a plurality of protein-intein fusion constructs into bacterial expression hosts using bacteriophage, and exploiting sexual reproduction in yeast to multiplicatively cross a diversity of protein-intein fusion constructs transformed into opposite mating types. Chimeric or recombinant proteins are produced according to the methods of the invention by introducing, most preferably by infection, one or more preferably a multiplicity of recombinant expression constructs into each cell, and then screening or more preferably selecting from cells expressing a desired phenotype. In a preferred embodiment, the present invention provides methods for producing proteins comprised of repeating sequence domains or motifs such as collagen and silk.

The methods of the present invention offer several advantages over prior methods. The inventive methods are not dependent on DNA sequence homology for recombination and thus permit production of hybrid proteins from distinct and unrelated genes. This is advantageous because conventional genetic recombination methods are dependent on the existence of regions of high sequence homology and thus bias the conventionally-produced recombinants for regions of high DNA sequence homology. This dependence on DNA sequence homology reduces the likelihood that protein domains that are capable of interacting and providing biological function but that share low DNA sequence homology will be produced using said conventional methods. In contrast, the methods of the present invention permit DNA sequence homology-independent hybrid proteins to be produced and either screened, or more preferably, selected for a desired, or more preferably unique, activity or phenotype. The inventive methods thus permit functional protein domain shuffling to be accomplished independent of any relatedness on a DNA sequence level, which is particularly useful in making chimeric proteins from fragments derived from different species.

An additional advantage of the methods of the invention over conventional techniques is that the inventive methods are not limited by size or transformation efficiencies. Typical methods for producing shuffled or domain-fused proteins exploit DNA technology to generate a plurality of genetic constructs. These constructs are introduced into expression hosts by transformation or transfection, thus the molecular diversity of the expressed protein ensemble is ultimately limited by the efficiency of the transformation or transfection process (i.e., the number of individual transformed clones that are generated). The inventive methods produce shuffled and/or domain fused proteins through the post-translational activity of trans inteins. As a result, constructs encoding pieces of the final product can be transformed or transfected individually, then efficiently co-localized in a common host cell through methods with greater efficiency than transformation or transfection (for example, infection with recombinant retrovirus or phage, or mating). Once co-localized, the trans intein elements promote recombination of the protein domains or fragments into contiguous polypeptides, with a theoretical diversity equal to the cross of the transformation and/or transfection efficiencies of the individual components.

The present invention also provides methods for producing trans-inteins from cis-inteins. Only one naturally-occurring trans-intein was known in the prior art (Hu et al., 1998, Proc. Natl. Acad. Sci. USA 95: 9226-9231). In this aspect, the invention provides genetically-engineered trans-inteins produced from cis-inteins using a modification of the ITCHY (incremental truncation for the creation of hybrid enzymes) technique, as described in co-owned and co-pending U.S. application Ser. No. 09/575,345, filed May 19, 2000, U.S. application Ser. No. 09/718,465 filed Nov. 15, 2000, and International Application No, PCT/US00/32114 filed Nov. 16, 2000, each of which is explicitly incorporated by reference herein.

Specific preferred embodiments of the present invention will become evident from the following more detailed description of certain preferred embodiments and the claims.

### BRIEF DESCRIPTION OF THE DRAWINGS

FIG. 1 is a schematic diagram of fusion strategies for the creation of hybrid proteins. Two different strategies, genetic fusion and fragment complementation, for the production of hybrid proteins are outlined. Genetic fusion is the conventional method for protein engineering. Exons are fused at the DNA level, transcribed and then translated as a hybrid protein. Fragment complementation occurs when proteins fragments associate spontaneously into hetero-oligomers, or their association is driven by oligomerization-directing domains. When trans-inteins serve as oligomerizing domain(s), the activity of the trans-intein generates contiguous polypeptides rather than hetero-oligomeric proteins. Trans-intein components are fused with coding sequence such as exons. The exon/intein fusions are transcribed, and translated. The resulting protein products associate and recombine as a hybrid protein via the interaction of complementary intein components.

FIG. 2 is a schematic diagram illustrating methods to engineer trans-inteins from cis-inteins. Nucleic acid encoding a cis-intein (SspDnaB) that has been inserted in the body of a nucleic acid encoding green fluorescent protein (GFP) is broken into two overlapping fragments so that the truncation target region (encoding the endonuclease or “endo” domain) is present in both constructs. Exonuclease III digestion to produce the truncated fragments is shown, followed by introduction of a multiplicity of fragments into recombinant cells and selection of GFP-producing cells by FACS.

FIG. 3 illustrates the results of FACS analysis of trans-inteins produced as shown in FIG. 2.

FIG. 4 shows the results of western blot analysis of intein-mediated protein production. Green fluorescent protein (GFP) is shown in lanes 3 and 8 expressed from plasmids pDIMC8 and pDIMN2, respectively. Each plasmid has a different origin of replication and encodes different antibiotic resistance genes as described in Ostermeier et al., 1999, Nature Biotech. 17: 1205-09. N-inteins from Ssp DnaB (IₙB) and Ssp DnaE (IₙE) trans-inteins were fused to genes encoding the amino terminus of GFP; C-inteins from Ssp DnaB (I_{c}B) and Ssp DnaE (I_{c}E) trans-inteins were fused to genes encoding the carboxyl terminus of GFP. IₙB runs as a doublet because it has an amber stop codon that is partially suppressed (lane 1, 4 & 6). Neither I_{c}B (lane 2) nor I_{c}E (lane 9) are observed when expressed alone (presumably because they are degraded). Homologous pairs (IₙB and I_{c}B; IₙE and I_{c}E) associate, as shown in lanes 4 and 7, respectively. In the presence of the N-intein, the C-intein fragments are protected. Moreover, both homologous pairs are functional protein ligases as shown by the production of full-length green fluorescent protein. With the heterologous pairs (IₙE and IₙB, and 1_{c}B and 1_{c}E) no evidence for ligase activity is apparent although one of the heterologous pairs does appear to associate weakly (as shown by the ability of IₙB to partially protect I_{c}E from degradation; compare lanes 6 and 9).

FIG. 5 is a demonstration that multiple trans-inteins operate independently in transfected cells. In these experiments, cells were transfected with various combinations of I_{C}B, I_{N}B, I_{C}E and I_{N}E fused to GFP reporter gene. The top row shows 40× brightfield illumination microscopy of cells transfected with intein components. The middle row shows 40× darkfield illumination microscopy of cells transected with intein components. The bottom row shows the results of FACS analysis of cells transformed with intern components. Cells transfected with homologous trans-intein components DnaBI_{C}/DnaBI_{N} (1^{st} panel) and DnaEI_{C}/DnaEI_{N} (4^{th} panel) exhibited significant fluorescence (67.4% and 39.7%, respectively). Cells transfected with non-homologous intein components DnaBI_{C}/DnaEI_{N} (2^{nd} panel) or DnaEI_{C}/DnaBI_{N} (3^{rd} panel) did not show significant fluorescence (12% and 13.4%, respectively).

FIG. 6 is a schematic diagram showing a strategy for trans-intein mediated polymerization of protein domains. The V5 epitope is fused to both I_{C}B and I_{N}B (upper left hand corner). This construct, termed BVB, would cyclize when homologous trans-intein components associate. Similarly the His-6 epitope is fused to both I_{C}E and I_{N}E (upper right hand corner). This construct, termed EHE, would cyclize when homologous trans-intein components associate. The V5 epitope is also fused to both I_{C}B and I_{N}E and termed BVE (lower left hand corner). The His-6 epitope also is fused to both I_{C}E and I_{N}B and termed EHB (lower right hand corner). When BVE or EHB are expressed alone, they fail to interact and cyclize. When expressed together bicyclic or polymeric products result.

FIG. 7 illustrates by Western analysis the results of BVE and EHB co-expression. Detection was based on His-tagged constructs with an anti-His antibody. For both blots, the far left lane (lane 1) is uninduced cells, followed by arabinose induced cells in lane 2 (0.5%). At the far right (lane 10) are cells induced with 1 mM IPTG. Lanes 3-9 show co-induction with 0.5% arabinose and IPTG at concentrations of 1 μM (lane 3), 3 μM (lane 4), 10 μM (lane 5), 30 μM (lane 6), 100 μM (lane 7), 300 μM (lane 8) and 1 mM (lane 9).

FIG. 8 illustrates by Western analysis that the BVB construct was spliced (FIG. 8, left blot, lane 2) and that EHB is appropriately processed when co-expressed with BVE (left blot, lane 7). BVE showed auto-processing (right blot, column 4). Based on the anti-His blot (left blot), only 2 of the four constructs had intact His-tags-BVB and EHE. BVE and EHE blot poorly because their His-tags are corrupted. The His tag on BVB provided some evidence that the construct was spliced. There was also clear evidence for splicing with the EHE construct despite the poor His epitope. The western blot indicated that there was little (if any) processing when EHB was expressed alone, but that there was detectable processing when co-expressed. If, as suggested from polyacrylamide gel electrophoresis (PAGE) results, the BVB construct was hampered by the N-terminal His-tag, rescue of activity by removal of the tag should have enhances product formation. The anti-V5 blot (right) indicated BVE autoprocessing, which appears to be slight albeit not zero. This was an unexpected, since the prior trans-splicing experiment showed that the DnaE N-intein could not protect the DnaB C-intein from degradation. The bottom of the figure includes mass spectral data.

FIG. 9 is a schematic diagram illustrating a method for using trans-inteins to engineer multidomain proteins. As shown in the Figure, engineering a modular protein from N domains or libraries of domains require (N−1) trans-inteins. To ensure assembly of each domain in the correct order in the primary sequence requires that each trans-intein interact exclusively with its homologous partner (e.g., aₙ for a_{c}, bₙ for b_{c}, (n−1)_{c}, for (n−1)_{c}, etc.) and not display promiscuity towards non-homologous partners (e.g., aₙ for b_{c}, bₙ for (n−1)_{c}, etc.) by virtue of the ability of multiple trans-intein to generate multiple crossovers.

FIG. 10 shows the amino acid sequence of two forms of silk useful for producing polymer proteins using trans-inteins.

FIG. 11 is a schematic diagram for producing biopolymers using trans-inteins.

FIG. 12 is a schematic diagram of methods of in vitro polymerization using trans-inteins for producing repeating protein polymers in vitro. Monomer (Y) functionalized with a trans-intein component is immobilized to a solid support. Addition of a fusion protein in which the next monomer of the desired polymeric protein (X) is embedded between the partner to the immobilized intein component (chevron) and a heterologous intein component (circle) for interaction with the subsequent functionalized monomer. Extension proceeds through the addition of the next functionalized monomer (Y), which is embedded between the partner to the immobilized intein component (diamond). Repeating this process leads to a polymer (poly-XY).

### DETAILED DESCRIPTION OF THE PREFERRED EMBODIMENTS

All references, patents and patent applications are hereby incorporated by reference in their entirety.

Within this application, unless otherwise stated, the techniques utilized may be found in any of several references known in the art, including but not limited to: Molecular Cloning: A Laboratory Manual, 3^{rd} Ed. (Sambrook, et al., 2001, Cold Spring Harbor Laboratory Press: New York); Gene Expression Technology (Methods in Enzymology, Vol. 185, Goeddel, ed., Academic Press, San Diego, Calif., 1991); “Guide to Protein Purification” in Methods in Enzymology (Deutshcer, ed., (1990) Academic Press, Inc.); PCR Protocols: A Guide to Methods and Applications (Innis et al., 1990, Academic Press, San Diego, Calif.); Culture of Animal Cells: A Manual of Basic Technique, 2^{nd} Ed. (Freshney, 1987, Liss, Inc. New York, N.Y.); Gene Transfer and Expression Protocols, pp. 109-128, Murray, ed., The Humana Press Inc., Clifton, N.J.), and the Promega 1996 Protocols and Applications Guide, 3^{rd} Ed. (Promega, Madison, Wis.).

In one aspect, the present invention provides a method for generating trans-inteins from cis-inteins comprising the steps of:

i) inserting into a first nucleic acid that encodes a protein a second nucleic acid comprising a nucleotide sequence encoding a cis-intein comprising an amino terminal portion (N-intein) and a carboxyl-terminal portion (C-intein) separated by a linker domain;

ii) breaking the cis-intein into two overlapping fragments, wherein the first fragment comprises a portion of the intein extending from the 5′ end of the intein through the 3′ end of the linker domain and wherein the second fragment comprises a portion extending from the 5′ end of the linker domain through the 3′ end of the intein;

iii) performing incremental truncation of each of the N-intein and the C-intein to produce every combination of deletion within the linker domain;

iv) performing intramolecular blunt-ended ligation to produce an N-intein truncation library and a C-intein truncation library wherein each truncation fragment comprising the library terminates translation of protein fragments encoded thereby on stop codons in all reading frames in N-inteins and initiates translation with a start codon from C-inteins;

v) introducing both libraries into a suitable host cell and

vi) selecting said host cells for trans-intein activity by detecting production of the protein encoded by the first nucleic acid.

In a preferred embodiment, the DNA sequence encoding a protein is a reporter gene. The reporter gene may be any known in the art including but not limited to beta-galactosidase, beta-glucoronidase, luciferase, and chloramphenicol acetyltransferase, and most preferably green fluorescent protein (GFP).

In a further embodiment, trans-intein activity is determined by reporter gene activity or the detection of a reporter gene itself. Reporter gene activity may be determined by growth (i.e., using a selection protocol), or biochemical activity, or a biophysical signal such as fluorescence, photon emission, change in color spectrum, transfer of radioactive groups, or by binding to an antibody and detected either directly or indirectly, for example, by conjugation to a detectable marker such as horseradish peroxidase or a fluorescent agent.

In an additional embodiment, trans-intein activity comprises an intein component interacting exclusively with a homologous intein partner. Trans-intein activity includes polymerization or cyclization of protein domains mediated by said trans-intein components.

As used herein, the term “intein” is intended to mean an internal peptide sequence of a protein precursor that is spliced out by transpeptidation during posttranslational processing to form a mature protein. The peptide sequences that are spliced together are termed exteins. The terminology is analogous to that in mRNA splicing, i.e. introns and exons.

As used herein, the term “cis-intein” is intended to mean a construct in which the intein and mature peptide or protein elements are expressed on the same precursor fusion protein.

As used herein, the term “trans-intein” is intended to mean an intein that is composed of two elements on separate polypeptides. These may occur naturally (for example, as disclosed in Wu et. al, 1988, Proc. Natl. Acad. Sci. USA 95: 9226-31) or be the products of genetic or protein engineering (Shingledecker et. al., 1998, Gene 207: 187-95, Southworth et. al., 1998, EMBO J. 17: 918-26, Wu et. al., 1998, Biochem. Biophys. Acta 1387: 422-32, Yamazaki et. al., 1998, J. Am. Chem. Soc. 120: 5591-2, and Ozawa et al., 2001, Anal. Chem. 73: 2516-2521). These elements must associate to affect transpeptidation.

As used herein, the terms and phrases “intein components” or “components of trans-inteins” is intended to mean polypeptides that must associate to affect intein-mediated transpeptidation.

As used herein, the term “N-intein” refers to an amino acid sequence corresponding to that found at the amino-terminus of an intein. As used herein, the term “C-intein” refers to an amino acid sequence corresponding to that found at the carboxyl-terminus of an intein. As used herein, the term “linker domain” refers to an amino acid sequence occurring between the N-intein and C-intein portions of an intein. The “linker domain” may also include some or all of the amino acid sequence corresponding to the adjacent N- and/or C-inteins.

For the purposes of this invention, the term “operably linked” in intended to indicate that the nucleic acid components of the inteins and intein-protein domain fusions of the invention are linked, most preferably covalently linked, in a manner and orientation that the nucleic acid sequences are under the control of and respond to the transcriptional, transcriptional, replication and other control elements comprising the vector when introduced into a cell.

In another aspect, the present invention provides a method for producing a recombinant multidomain protein comprising one or a plurality of protein domains covalently linked together, the method comprising the steps of:

i) fusing each of one or a multiplicity of nucleic acids encoding a polypeptide, polypeptide fragments, or protein domains to a trans-intein component to produce a plurality of intein-domain fusion fragments;

ii) ligating each of a plurality of intein-domain fusion fragments to an expression vector;

iii) introducing a plurality of said vectors containing the intein-domain fusion fragments into a suitable host cell;

iv) expressing the plurality of intein-domain fusion fragments to generate a plurality of fusion proteins;

v) screening or selecting the host cells to detect

vi) subjecting host cells to selections or screen to identify cells containing recombinant multidomain proteins comprising one or a plurality of protein domains covalently linked together.

In a further aspect, the invention provides libraries of chimeric multidomain proteins produced by the method described above. In a preferred embodiment, hybrid protein libraries are produced by introducing multiple vectors containing domain/intein fusions (truncation libraries of Example 1) into host cells and allowing the subsequent post-translational polymerization of domain/intein fusions into chimeric proteins via the actions of trans-inteins.

The present invention provides host cells transfected with vectors comprising the domain/intein fusions described herein. As used herein, the term “vector” refers to a nucleic acid molecule capable of transporting, replicating and/or expressing another nucleic acid to which it has been linked. One type of vector is a “plasmid,” which is known in the art to mean a circular double stranded DNA into which, inter alia, additional DNA segments may be cloned. Another type of vector is a viral vector, whereby, inter alia additional DNA segments may be cloned into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors), are obliged to be integrated into the genome of a host cell upon introduction into said host cell, and thereby are replicated along with the host genome. Moreover, certain vectors are capable of directing the expression of genes to which they are operably linked. Such vectors are referred to herein as “recombinant expression vectors” or simply “expression vectors”. In the present invention, the expression of the domain/intein fusion polypeptide sequence is directed by the promoter sequences of the invention, by operably linking the promoter sequences of the invention to the gene to be expressed. In general, expression vectors useful in the recombinant DNA arts are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The vector may also contain additional sequences, such as a polylinker for subcloning additional nucleic acid sequences, preferably a polylinker comprising one or multiplicity of restriction enzyme recognition sites and most preferably a polylinker comprising one or multiplicity of restriction enzyme recognition sites uniquely present in the polylinker, transcriptional splice signals to facilitate expression and processing of a transcript in mammalian cells, or a polyadenylation signal to effect proper polyadenylation of the transcript. The nature of the polyadenylation signal is not believed to be crucial to the successful practice of the invention, and any such sequence may be employed, including but not limited to the SV40 and bovine growth hormone poly-A sites. Also contemplated as an element of the vector is a termination sequence, which can serve to enhance message levels and to minimize read through from the construct into other sequences. Additionally, expression vectors typically have selectable markers, often in the form of antibiotic resistance genes, that permit selection of cells that carry these vectors.

The present invention provides host cells transfected with vectors comprising the domain/intein fusions described herein. As used herein, the term “host cell” is intended to refer to a cell into which a nucleic acid of the invention, such as a DNA sequence encoding a protein fused to a trans-intein component (domain/intein construct), has been introduced. Such cells may be prokaryotic, which can be used, for example, to produce large amounts of the chimeric proteins of the invention, or the cells maybe eukaryotic useful, inter alia for functional studies.

The host cells can be transiently or stably transfected with one or more of the domain/intein constructs of the invention. Such transfection with one or more of the expression vectors of the invention can be accomplished by any method known in the art, including, but not limited to bacterial transformation methods, calcium phosphate co-precipitation, electroporation, or liposome mediated-, dextran mediated-, polycationic mediated-, or viral mediated transfection. See, for example, Sambrook et al., 2002, Id.; Freshney, 1987, Id.

Multiple domain/intein fusion vectors can be transfected into host cells to produce a “library” of fusion proteins. These libraries may contain sequences from families of related genes or sequences from distinct and unrelated genes.

The terms “hybrid protein”, “fusion protein”, and “recombinant multidomain protein” are used interchangeably. In a preferred embodiment, hybrid proteins are comprised of one or more protein domains, fragments or epitopes, fused together post-translationally via the actions of trans-inteins. In an additional embodiment, products resulting from trans-intein mediated fusion can be cyclic (circular) or polymeric (linear). Protein products may contain one or preferably more than one protein domain fused together post-translationally. Trans-intein mediated fusion may be intracellular, or in vitro, for example, in cell culture medium. Domain/intein monomers may be isolated or secreted from cells and allowed to polymerize in vitro.

In another aspect, the invention provides a method for making proteins comprised of repeating protein polymers comprising,

1) fusing each of one or a multiplicity of nucleic acids encoding a first monomeric component of the protein polymer to a C-intein and N-intein of two different trans-inteins to produce a plurality of intein-domain fusion fragments;

2) ligating each of a plurality of intein-domain fusion fragments to an expression vector;

3) expressing the plurality of intein-domain fusion fragments to generate a plurality of fusion proteins;

4) screening or selecting the host cells to identify cells containing recombinant multidomain proteins comprising one or a plurality of protein domains covalently linked together.

In preferred embodiments, the intein-fused monomeric components are expressed in the same host cell and the polymeric protein product harvested therefrom. In alternative preferred embodiments, each monomeric component is expressed in a distinct host cell, the monomers purified therefrom and then combined in an appropriate reactor to enable trans-intein mediated polymerization in vitro. In yet alternative preferred embodiments, each monomeric component is expressed in a host cell and is secreted from said host cells and then combined together in an appropriate reactor to enable trans intein mediated polymerization in vitro.

In a preferred embodiment, the repeating polymeric protein is silk, collagen, or laminin. The methods of the present invention are also useful for the production of other naturally repeating proteins known in the art. The term “repeating protein polymer” refers to proteins comprised of repeating units of specific amino acid sequence motif.

The term “reactor” refers to a container such as a test tube, microfuge tube, or other container suitable for in vitro trans-intein mediated polymerization. The reactor may also include a suitable living host cell.

The present invention may be better understood with reference to the accompanying examples that are intended for purposes of illustration only and should not be construed to limit the scope of the invention, as defined by the claims appended hereto.

### EXAMPLES

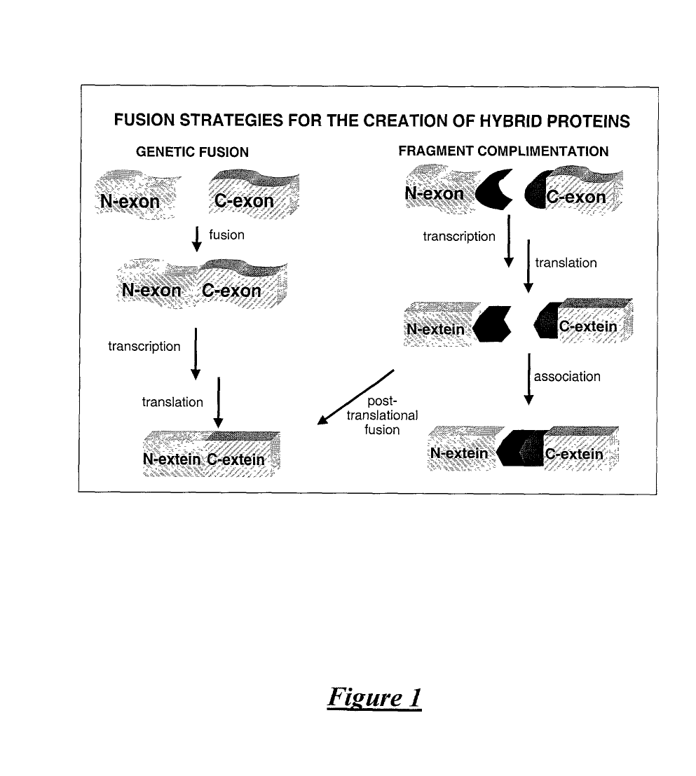

A method for producing hybrid proteins via the action of modified trans-inteins according to the invention is illustrated schematically in FIG. 1. Coding sequences were fused to complementary components of trans-inteins, introduced into a suitable host cell, transcribed and translated. The fusion proteins associated post-translationally resulting in the production of hybrid proteins, as described more fully in the Examples below.

### Example 1

### Production of Trans-Inteins from Cis-Inteins

The production of chimeric peptide libraries from discrete polypeptide sequences was mediated by the activity of trans-inteins. Novel trans-inteins were engineered from cis-inteins utilizing the ITCHY (incremental truncation for the creation of hybrid enzymes) method as described below and in co-owned and co-pending U.S. application Ser. No. 09/575,345, filed May 19, 2000, U.S. application Ser. No. 09/718,465 filed Nov. 15, 2000, and International Application No, PCT/JS00/32114 filed Nov. 16, 2000, incorporated by reference hereon. All polymerases, restriction enzymes and endo- and exonucleases were obtained from New England Biolabs (Waltham, Mass.) or an equivalent vendor and used according to manufacturer's instructions.

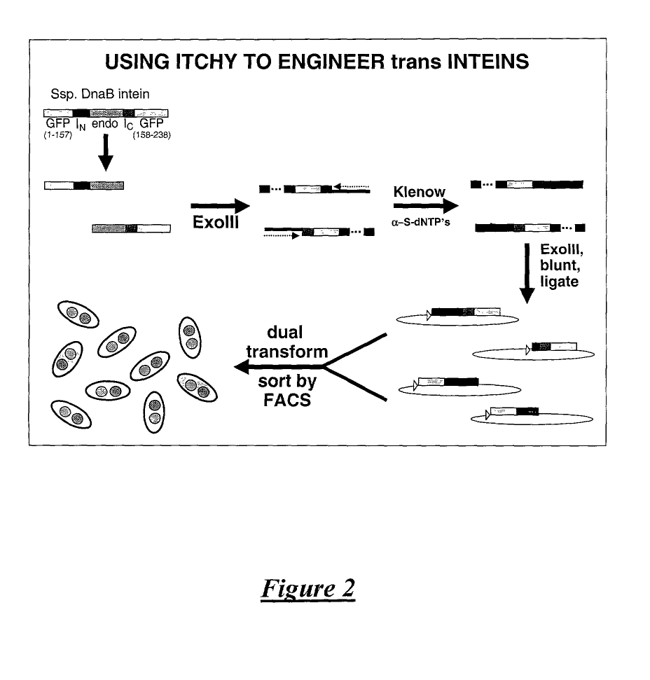

Two separate protein domains of a reporter gene, enhanced green fluorescent protein (eGFP, Clontech), were recombined into a functional reporter using a modified trans-intein. The gene encoding the cis-intein, SspDnaB (Wu et al., 1998, Biochim. Biophys. Acta 1387: 422-32), incorporated herein by reference, was inserted into the GFP gene between codons for amino acids 157 and 158. This location in eGFP was chosen because it has been shown that the resulting fragments of GFP have little or no affinity for one another unless fused to high affinity dimerizing domains (Ghosh et. al., 2000, J. Amer. Chem. Soc. 122: 5658-9). The resulting chimeric eGFP 1-157/SspDnaB intein/eGFP 158-238 gene was used as a target for amplification by the polymerase chain reaction (PCR). Four primers were designed to generate two PCR products: one encompassing sequence encoding eGFP residues 1-157 and the N-intein and endonuclease domains of the SspDnaB intein, and a second consisting of sequence encoding the endonuclease domain and C-intein from the SspDnaB intein and eGFP residues 158-238 (see FIG. 2). Primers annealing to intein/endonuclease domain boundaries were designed by analogy with Wu et. al. (Id.). Primers annealing to the 5′-end of the GFP gene were designed with SphI and NdeI restriction sites. Primers annealing at the 3′-end of the GFP gene were designed with PstI and SacI restriction sites. Restriction sites were chosen to direct the incremental truncation process and ensure efficient, orthogonal cloning of the processed inserts as described below. Libraries were generated by random incorporation of α-thio-dNTP's into PCR products amplified from the chimeric template with appropriate primer pairs. An optimal ratio (100:1) of dNTP's to α-thio-dNTP's (300 μM total in reaction mixture) was determined empirically and incorporated on average one α-thio-dNTP every 800 bases, which was appropriate to yield deletion libraries that scan the entire SspDnaB endonuclease domain. Truncation libraries were resolved through the action of exonuclease III (ExoIII) on PCR products. ExoIII cannot digest past α-thio-dNTP's incorporated in the DNA backbone. Assuming that α-thio-dNTP's were randomly distributed throughout a region of interest, exhaustive treatment with ExoIII (120 U/μg, 30 min., 37° C.) resulted in a complete library in which every single base deletion was represented. The end of the PCR product encoding the reporter gene was protected from digestion with primer encoded restriction endonucleases (SphI on the PCR product containing the 5′-end of the eGFP gene and PstI on the PCR product containing the 3′-end of the eGFP gene). These enzymes generated 5′-recessed ends that were not substrates for ExoIII thereby directing ExoIII activity to the scanning region of interest. Following ExoIII digestion, PCR fragment libraries were treated with Mung Bean endonuclease to remove single stranded overhangs, and with Klenow fragment to generate blunt ends (as per Lutz et al., 2001, Nucleic Acids Res. 29: E16). Libraries of blunted-ended fragments were then subjected to a second round of restriction digestion (using NdeI on library fragments encoding the 5′-end of the eGFP reporter gene and SacI on library fragments encoding the 3′-end of the eGFP reporter gene) to enable orthogonal cloning into suitable vectors (pDIM-N2 for fragments encoding the 5′-end of the eGFP reporter gene; pDIM-C8 for fragments encoding the 3′-end of the eGFP reporter gene, see Ostermeier et al., 1999, Proc. Nat'l. Acad. Sci. USA 96: 3562-3567). Cloned library fragments were then co-transformed into bacteria, and cells containing intein fragments able to associate in trans were isolated by fluorescence activated cell sorting (FACS).

This method resulted in trans-inteins with improved properties (such as activity in heterologous hosts, soluble activity in vitro, desired kinetic profiles to allow intracellular targeting, activity under desired environmental conditions such as pH or redox potential to give maximal activity upon delivery to desired intracellular organelle) as disclosed in Example 2 below.

### Example 2

### Detection of Trans-Intein Activity

Fluorescence activated cell sorting (FACS) analysis was used to detect transformed cells that expressed functional hybrid proteins as follows.

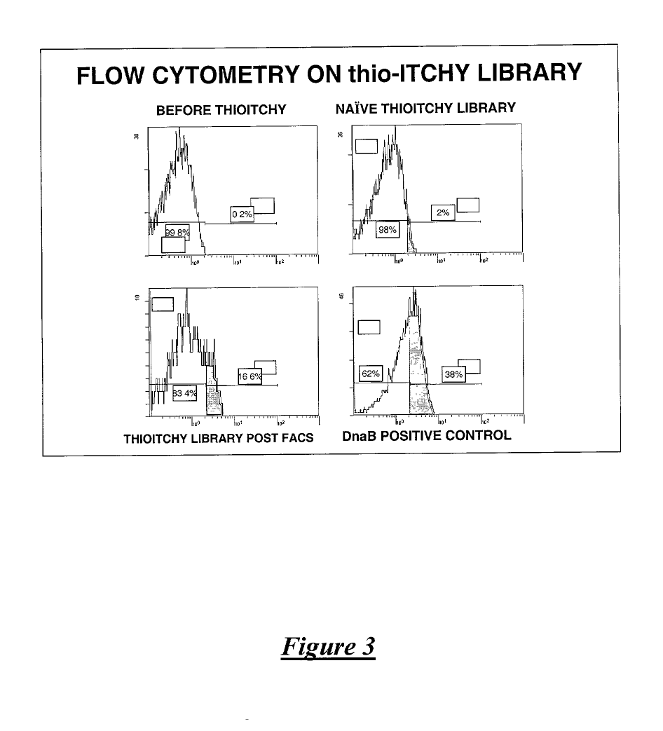

The trans-intein truncation libraries described above in Example 1 were transformed into E. coli. The association of an intein I_{N} component with a complementary intein I_{C} component resulted in trans-intein mediated fusion of the reporter gene, green fluorescent protein. Specific fluorescence at 510 nm was observed in cells that underwent successful trans-intein mediated reporter gene fusion. The results of these experiments, showing functional trans-intein activity by reporter gene fluorescence are shown in FIG. 3. In the panel labeled “Before THIOITCHY,” two parental plasmids each with the entire intact endonuclease domain were transformed into the same E. coli host and analyzed by FACS. Little or no fluorescence was observed in this analysis. Following thiol-ITCHY (shown in the “naïve THIOITCHY panel”) the number of particles in the fluorescence gate increased by an order of magnitude. The fluorescent population was also further enriched by fluorescence activated cell sorting. After a single round (shown in the “THIOITCHY library post-FACS” panel) the fluorescence in the remaining library was almost half (16.6%) of the fluorescence (38%) of an isogenic control construct known to be a functional trans-intein (shown in the “DnaB positive control” panel; see Wu et. al., 1998, Id.).

These results clearly demonstrate that ITCHY is useful for engineering new trans-inteins from existing cis-inteins.

### Example 3

### Polymerization of Hybrid Proteins with Multiple Independent Trans-Inteins

The capacity of trans-inteins to produce hybrid proteins was also analyzed by detecting hybrid proteins. Libraries of chimeric proteins were recombined at the post-translational level through the association of modified homologous trans-inteins partners. Engineered trans-inteins demonstrated fidelity towards homologous partners as indicated by Western blot analysis. When linked to protein domains, these novel trans-inteins associated and polymerized protein fragments into cyclic and linear hybrid proteins.

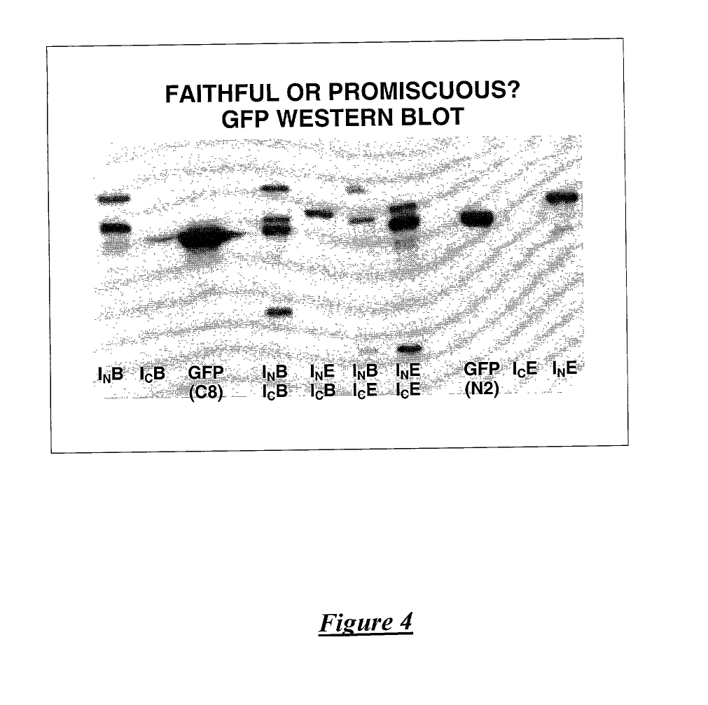

The results of Western blot analysis are shown in FIG. 4. N-inteins from Ssp DnaB (I_{N}B) and Ssp DnaE (I_{N}E) trans-inteins were fused to genes encoding the amino terminus of green fluorescence protein, and C-inteins from Ssp DnaB (I_{C}B) and Ssp DnaE (I_{C}E) trans-inteins were fused to genes encoding the carboxyl terminus of GFP. These constructs were introduced into cells either alone or in “faithful” and “promiscuous” combinations to determine the fidelity of intein activity. The I_{N}B fragment (lane 1) introduced by itself into a cell ran as a doublet because of an amber stop codon that is partially suppressed in the bacterial expression strain. I_{C}B and I_{C}E were not observed when expressed alone, presumably because they were degraded. Homologous pairs I_{N}B: I_{C}B and I_{N}E I_{C}E associated and produced a protein having a molecular weight consistent with full-length GFP. Both homologous pairs produced functional protein ligases as shown by the production of full length GFP. With the heterologous pairs (I_{N}E and I_{C}B; I_{N}B and I_{C}E), no evidence for ligase activity was apparent, although one of the heterologous pairs does appear to associate weakly (as shown by the ability of I_{N}B to partially protect I_{C}E from degradation). These results clearly demonstrate that at least these two trans-inteins show fidelity towards their homologous partners and should therefore be able to function independently in the presence of the other.

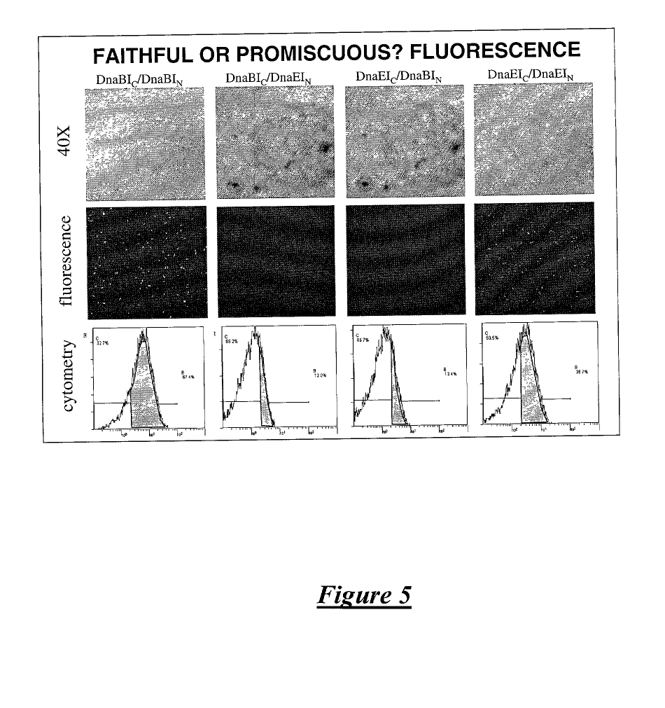

These results were confirmed in cells transfected with various combinations of I_{C}B, I_{N}B, I_{C}E and I_{N}E GFP reporter gene fusions. These results are shown in FIG. 5. DnaBI_{C}/DnaBI_{N} (1^{st} panel) and DnaEI_{C}/DnaEI_{N} (4^{th} panel) transfected cells exhibited fluorescence resulting from trans-intein fusion of the reporter gene, as shown by fluorescence detected by microscopy and cell sorting. In contrast, cells transfected with non-homologous intein components DnaBI_{C}/DnaEI_{N} (2^{nd} panel) or DnaEI_{C}/DnaBI_{N} (3^{rd} panel) did not show appreciable fluorescence, indicating no association between the non-homologous intein components.

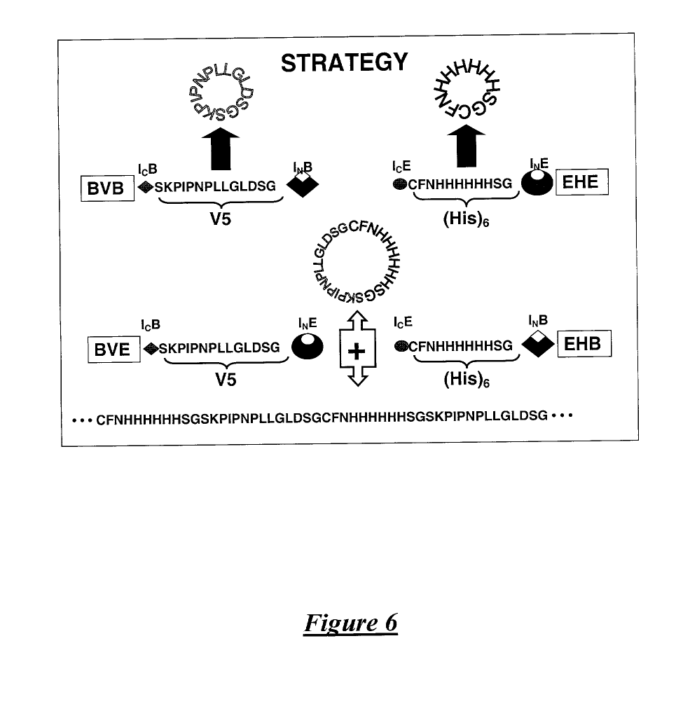

In other experiments, trans-splicing constructs produced results that suggested that DnaB & DnaE trans-inteins operate independently to recombine and produce chimeric proteins. Four constructs were generated: one with a V5 epitope bracketed by the C- and N-inteins from the DnaB trans-intein (FIG. 6, BVB, upper left hand corner); one with a His-6 epitope bracketed by the C- and N-inteins from the DnaE trans-intein (FIG. 6, EHE, upper right hand corner); one with a VS epitope bracketed by the DnaB C-intein and the DnaE N-intein (FIG. 6, BVE, lower left hand corner); and one with a His-6 epitope bracketed by the DnaE C-intein and the DnaB N-intein (FIG. 6, EHB, lower right hand corner). The extein residues between the epitopes and inteins were identical to the amino acids in the trans-splicing constructs described above. Constructs of the type EHE or BVB should yield cyclic products based on literature precedent (Scott et al., 1999, Proc. Natl. Acad. Sci. USA 96: 13638-43). Neither the BVE nor EHB constructs were expected to splice when expressed by themselves, since the intein components should fail to interact, and this behavior was in fact observed. When the two constructs were co-expressed, both inteins were expected to be active, and bicyclic (middle) or polymeric (bottom) products expected to, and did, result. Products were visualized in crude lysates by Western blotting polyacrylamide (PAGE) gels with both anti-V5 and anti-His antibodies. These Western blots suggested that BVE and EHB interact and underwent trans-intein mediated polymerization to produce a chimeric protein.

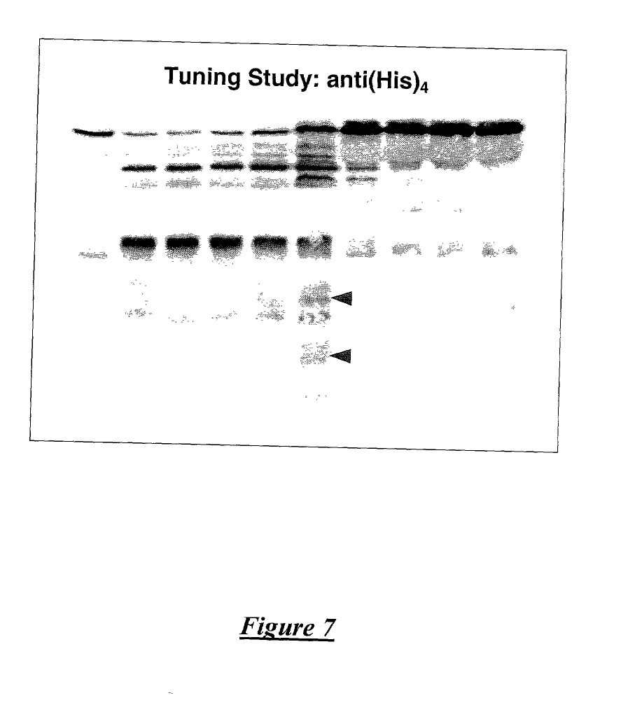

Further, BVE and EHB were cloned into inducible expression vectors, co-expressed and examined by Western analysis. BVE was cloned into pET28 with an N-terminal His-tag for antibody detection in Western blots and for subsequent purification. EHB was cloned into pAR (Perez-Perez et al., Gene 158:141-142) so that expression of the EHB fragment could be induced with arabinose independently of the induction of BVE with IPTG. The vectors encoding each piece were co-transformed into the expression strain, tuner-DE3 (Novagen), so that the induction of the BVE fragment could be better controlled. The results are shown in FIG. 7 (blot incubated with anti-His antibody).

In the anti-His Western blot (FIG. 7), at the far left (lane 1) is shown results from uninduced cells, followed by results arabinose induced cells in lane 2 (0.5%). At the far right (lane 10) are results from cells induced with 1 mM IPTG. Lanes 3-9 show results from cells subjected to co-induction with 0.5% arabinose and isopropylthiogalactoside (IPTG) at concentrations of 1 μM (lane 3), 3 μM (lane 4), 10 μM (lane 5), 30 μM (lane 6), 100 μM (lane 7), 300 μM (lane 8) and 1 mM (lane 9). These results provide clear evidence for the production of low molecular weight products when the concentration of IPTG is low (0-100 μM) with optimal product formation at IPTG concentration of about 30 μM. At an IPTG concentration of 100 μM, expression of BVE began to overwhelm the system This is shown in the Figure in lanes 7-10, where the His-tagged BVE fragment is the thick band just below the position of the uppermost band on the blot. Product formation occurs even when BVE is not induced (i.e., at an IPTG concentration of 0) because the pET vector “leaks” (the gray BVE band is clearly visible in lane 1).

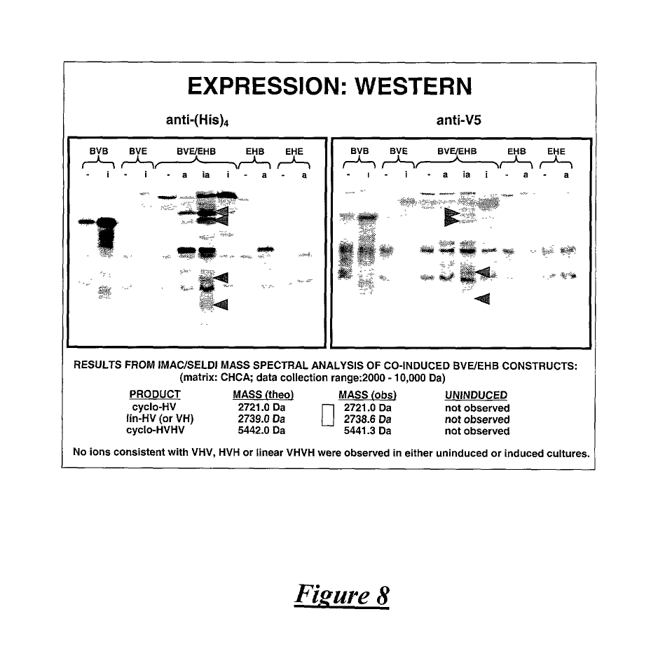

Tuner-DE3 cells (Novagen) transformed with expression vectors as shown in FIG. 8 were grown to an OD₆₀₀ of 0.4 and induced by the addition of either IPTG (labeled I in FIG. 8; 1 mM), arabinose (a; 0.5%), both IPTG and arabinose (ia; 30 μM IPTG; 0.5% arabinose) or neither (−) and incubated with shaking at 25° C. for 20 hr. Expression products were visualized by Western blot with antibodies to either (His)4 (QIAGEN) or V5 (Invitrogen). His tags were added to the amino terminus of constructs BVB and BVE to aid in visualization, however, point mutations in the His epitopes in constructs BVE and EHE significantly attenuated detection of these two constructs with anti(His)-4. Numerous unique products were apparent in both anti-V5 and anti-His-4 blots upon co-expression of BVE and EHB constructs (red arrows), indicating that these products contained both V5 and His-6 epitopes.

To show conclusively that products resulted from the concerted activity of both intein pairs, cells were lysed in an 8M urea solution in phosphate buffered saline (PBS+8M) and loaded onto an immobilized metal affinity chromatography (IMAC) surface enhanced laser desorption-ionization (SELDI) mass spectral analysis chip pre-equilibrated with nickel sulfate and lysis buffer. Chips were washed with buffer and increasing concentrations of imidazole, then rinsed with water, dried and subjected to mass spectral analysis using an alpha-cyano-4hydroxycinnamic acid matrix (which is optimally suited for analysis of low molecular weight peptides). Molecular ions consistent in mass with linear (HV) and cyclic (c-HV) mono-adducts and a cyclic di-adduct (c-HVHV) were observed in induced but not uninduced samples. These ions survived washes of 0 μM, 3 μM and 30 μM imidazole, but disappeared after treatment with 300 μM imidazole, consistent with the affinity of a His-6 tag for a Nickel IMAC surface.

These results demonstrated that modified trans-intein showed fidelity towards their homologous partners and suggested that modified trans-inteins function independently when co-expressed in a cell. In addition, modified trans-inteins were capable of inducing polymerization of separate protein domains.

### Example 4

### Combinatorial Libraries of Hybrid Proteins

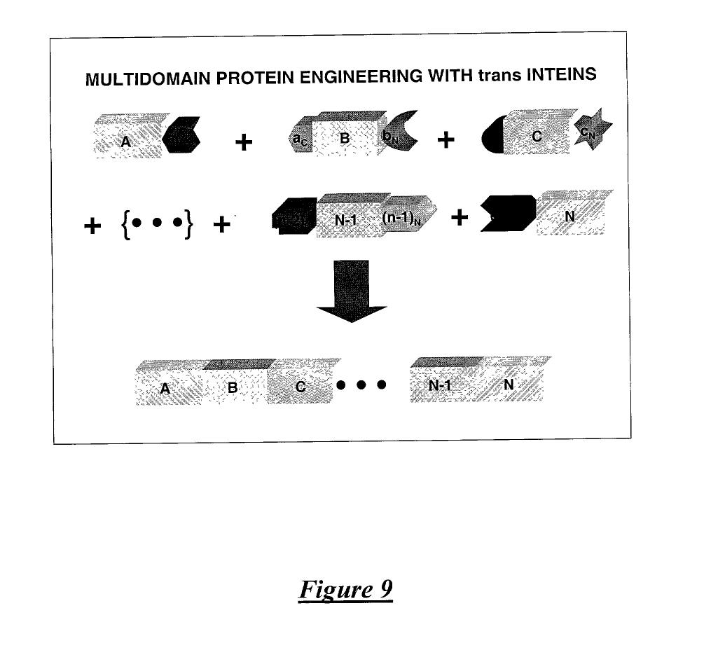

To extend intein-mediated polymerization beyond binary fusions, multiple trans-intein are required. Engineering a modular protein from N domains or libraries of domains generally requires (N−1) trans-inteins. Each protein domain or fragment is flanked at the 5′ and 3′ end by a trans-intein component (except for the first domain and last domain). This approach is shown schematically in FIG. 9. A protein domain is fused to trans-intein component A_{C} at it 5′ end and B_{N} at its 3′ end, and the following domain of the chimeric protein is fused to trans-intein component B_{C} at it 5′ end and C_{N} at its 3′ end, and so forth. The trans-intein component (I_{N}) on the 3′ end of a protein domain interacts with its homologous partner (I_{C}) fused to the 5′ end of the next protein domain. For example, A_{N} and A_{C}, B_{N} and B_{C}, and (n−1)_{N} for (n−1)_{C}, and so forth. In order for domains to arrange in the desired order within the engineered protein, each trans-intein must interact exclusively with its homologous partner and not with non-homologous partners.

FIG. 9 illustrates an alternative and entirely distinct mechanism from DNA shuffling for condensing beneficial mutations (i.e. from each domain library) onto a single polypeptide. Since domain boundaries are defined by the positions of trans-inteins, crossovers are not limited to occur in regions of high sequence homology, as is the case for DNA shuffling. Larger libraries are accessible by post-translational fusion than are possible in methods that depend upon the creation of chimeric genes (such as DNA shuffling or SCRATCHY) because intracellular recombination can generate libraries equal in size to the cross for the transformation efficiencies of all the individual domain libraries (see, for example, Ostermeier & Benkovic, 2000, J. Immunol Meth. 237:175-86). As library size increases the likelihood that clones containing all or many beneficial mutations on a single construct are represented in the library also increases. This method requires access to multiple trans-intein that can function independently in the presence of one another.

The utilization of trans-inteins permits multiple protein domains to be covalently linked to one another to produce a plurality of different hybrid proteins, and is not limited in any way to sequence homology, either at the nucleotide or amino acid level. In this way, protein domains even from unrelated genes having little or no sequence identity are produced by these methods.

The results shown in the Examples above indicated that it is possible to condense beneficial mutations from a multiplicity of domain libraries onto a single polypeptide, based on the ability of multiple trans-intein to covalently link multiple protein domains. For this approach to be optimally useful, large libraries are necessary to increase the likelihood of having beneficial mutations on a single construct represented in the library. The ability of post-translational fusion methods to generate libraries equal in size to the cross of the transformation efficiencies of all the individual domain libraries is a great advantage. This method offers an alternative and entirely distinct approach from DNA shuffling for accumulating positive mutations.

### Example 5

### Method for Producing a Repeating Protein Polymer

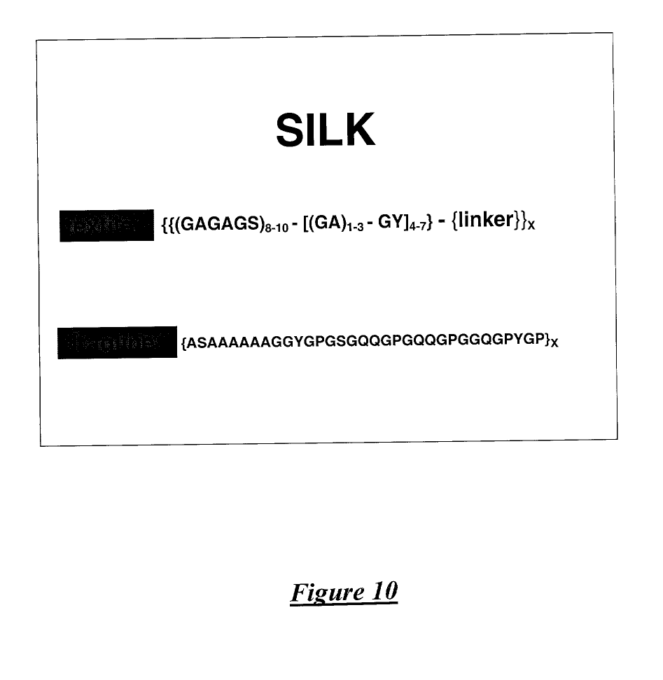

Another use for trans-inteins as provided by the invention is production of repeating protein polymers. Repeating protein polymers (such as silk or collagen) have proven refractory to standard recombinant production methods due to the repetitive nature of the desired product that requires a similarly repetitive gene (illustrated in FIG. 10). Such genetic constructs are unstable because they are prone to insertion, deletion, and recombination in host strains. The use of trans-inteins to generate such polymeric materials eliminates genetic instability since only a single monomer (or a limited number of monomers, as desired) needs to be encoded. Trans-inteins are known to function both in vitro and in vivo, thus trans-intein mediated polymerization would be possible either within cells or in vitro following the purification of monomeric starting material.

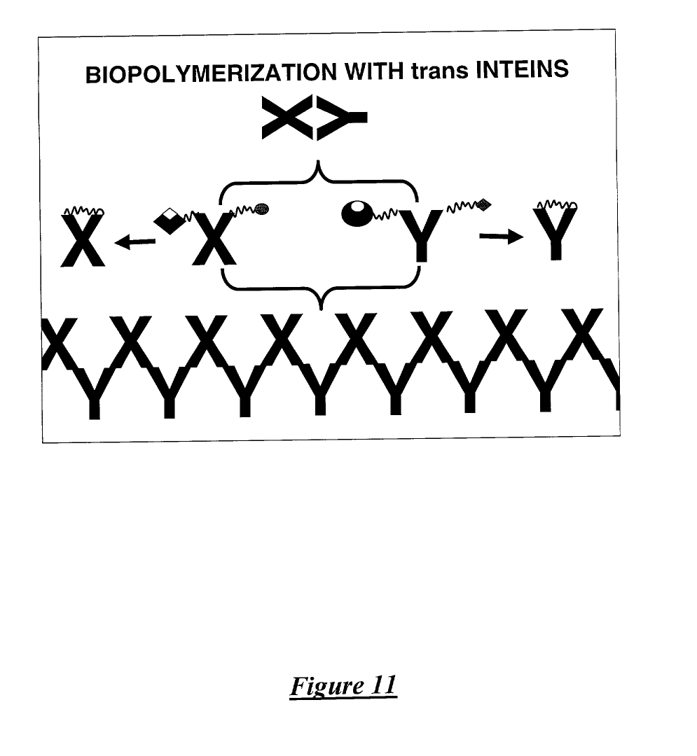

The trans-inteins used for making multimodular proteins or polymers must show fidelity because cyclization and polymerization are essentially the same process. An important difference is whether a reactive end is on the same molecule (cis-splicing leading to cyclization) or on a different molecule (trans-splicing leading to polymerization). Intramolecular cyclization predominates over bimolecular reactions such as polymerization when homologous intein pairs flank monomers of interest. For example, as shown in FIG. 11, if the chevron can interact with the circle, “X” will be cyclized (FIG. 11, arrow at left). Likewise, if the crescent interacts with the diamond, “Y” will be cyclized (FIG. 11, arrow at right). However, if the chevron interacts exclusively with the diamond, and the crescent interacts exclusively with the circle, bicyclic (FIG. 11, top) or polymeric (FIG. 11, bottom) products will result. The ratio of cyclization to polymerization depends on expression levels: at high expression levels polymerization should predominate over cyclization, with the converse being true at low expression levels. Control over partitioning to cyclic products is achieved by tuning the expression level of one monomer with respect to the other. By using multiple trans-inteins, block copolymers with several monomeric substituents were accessible. Even if polymers consisting of a single monomeric unit are desired (e.g., where X=Y), two trans-inteins are preferred because intramolecular cyclization is much more efficient than polymerization, especially when being catalyzed by trans-inteins (see, for example, Evans et al., 1999, J. Biol. Chem. 274:18359-63 and Scott et al., 1999, Proc. Natl. Acad. Sci. USA 96:13638-43). Trans-inteins are compatible for protein ligation both in vitro and in vivo, so polymerization is also possible either in vitro or in host cells.

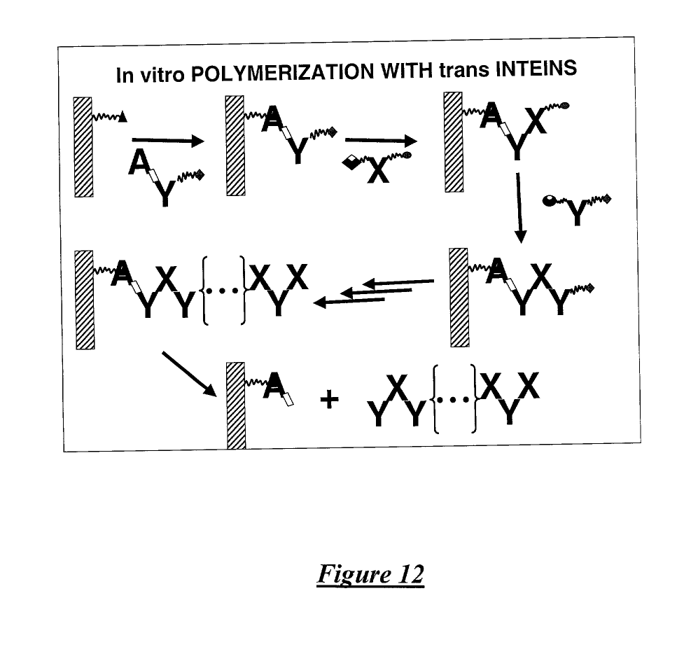

Trans-inteins that have activity in vitro, such as SspDnaE, can be used for cell-free synthesis of repeating protein polymers (as shown in FIG. 12). Synthesis of such polymers advantageously proceeds by a Merrifield-like process, where a monomer (Y) functionalized with a trans-intein component is immobilized to a solid support (striped bar) through the affinity of a receptor (A) for its ligand (triangle). Addition of a fusion protein in which the next monomer of the desired polymeric protein (X) is embedded between the partner to the immobilized intein component (chevron) and a heterologous intein component (circle) for interaction with the subsequent functionalized monomer. Extension proceeds through the addition of the next functionalized monomer (Y). which is embedded between the partner to the immobilized intein component (diamond). Repeating this process leads to a polymer (poly-XY), which is held to the solid support through the interaction of the reporter fused to the initial monomeric equivalent of the polymer with its column-bound ligand. The polymer can then be eluted from the column by competition for the receptor with soluble ligand, and/or can be cleaved from the receptor by introducing an appropriate cleavage site (yellow box).

It should be understood that the foregoing disclosure emphasizes certain specific embodiments of the invention and that all modifications or alternatives equivalent thereto are within the spirit and scope of the invention as set forth in the appended claims.

We claim:

1. A method for producing a recombinant multidomain protein comprising one or a plurality of protein domains covalently linked together, the method comprising the steps of;
a) fusing each of one or a multiplicity of nucleic acids encoding a polypeptide, polypeptide fragments, or protein domains to a trans-intein component to produce a plurality of intein-domain fusion fragments;
b) ligating each of a plurality of intein-domain fusion fragments to an expression vector;
c) introducing a plurality of said vectors containing the intein-domain fusion fragments into a suitable host cell;
d) expressing the plurality of intein-domain fusion fragments to generate a plurality of fusion proteins;
e) selecting or screening host cells to identify cells containing recombinant multidomain proteins comprising one or a plurality of protein domains covalently linked together.

2. A library of fusion proteins comprising a plurality of host cells expressing recombinant multidomain proteins comprising one or a plurality of protein domains covalently linked together and produced by the method of claim 1.

3. A recombinant multidomain protein comprising one or a plurality of protein domains covalently linked together and produced by the method of claim 1.

4. A method for making a polymeric protein comprising repeating protein polymer motifs, the method comprising the steps of:
i) ligating a plurality of n genes for n monomeric components of the polymeric protein between a C-intein and an N-intein of two different trans-inteins, wherein the C-intein of the nth gene specifically interacts with the N-intein of the (n−1)^{th} monomeric component to covalently link the nth gene product with the (n−1)^{th} gene product;
ii) ligating each of a plurality of the gene-intein ligation products of step (i) to an expression vector;
iii) introducing a plurality of said vectors containing the gene-intein ligation products into a suitable host cell;
iv) expressing the plurality of gene-intein ligation products in the host cell; and
v) isolating the harvesting the polymeric protein therefrom.

5. The method of claim 4, wherein each gene-intein ligation product is expressed in a different host cell and the products combined and the polymeric protein polymerized in vitro.

6. The method of claim 4, wherein each gene-intein ligation product is expressed in a different host cell and secreted from said host cells and wherein the secreted products combined and the polymeric protein polymerized in vitro.

7. The method according to claim 4 wherein the polymeric protein comprises a plurality of monomeric protein domains covalently liked together.

8. The method of claim 4 wherein each of the monomeric protein domains is the same.

9. The method of claim 4 wherein the monomeric protein domains are different.

10. The method of claim 8, wherein the polymeric protein is silk, collagen or laminin.

11. A method for generating a trans-intein from a cis-intein comprising the steps of:
i) inserting into a first nucleic acid that encodes a protein a second nucleic acid comprising a nucleotide sequence encoding a cis-intein comprising an amino terminal portion (N-intein) and a carboxyl-terminal portion (C-intein) separated by a linker domain;
ii) breaking the cis-intein into two overlapping fragments, wherein the first fragment comprises a portion of the intein extending from the 5′ end of the intein through the 3′ end of the linker domain and wherein the second fragment comprises a portion extending from the 5′ end of the linker domain through the 3′ end of the intein;
iii) performing incremental truncation of each of the N-intein and the C-intein to produce every combination of deletion within the linker domain;
iv) performing intramolecular blunt-ended ligation to produce an N-intein truncation library and a C-intein truncation library wherein each truncation fragment comprising the library terminates translation of protein fragments encoded thereby on stop codons in all reading frames in N-inteins and initiates translation with a start codon from C-inteins;
v) introducing both libraries into a suitable host cell and
vi) selecting said host cells for trans-intein activity by detecting production of the protein encoded by the first nucleic acid.

12. The method of claim 11, wherein said first nucleic acid encodes a reporter gene.

13. The method of claim 12, wherein trans-intein activity is detected by reporter gene activity.

14. The method of claim 11, wherein trans-intein activity comprises an intein component interacting exclusively with a homologous intein partner.
